(19) United States
(12) Patent Application Publication
(10) Pub. No.: US 2020/0085362 A1
(43) Pub. Date: Mar. 19, 2020

(54) METHOD OF EVALUATING A PSYCHOPHYSIOLOGICAL STATE OF A PERSON

(71) Applicant: OBSHCHESTVO S OGRANICHENNOJ OTVETSTVENNOST`YU "MNOGOPROFILNOE PREDPRIYATIE "ELSYS", St. Petersburg (RU)

(72) Inventor: Viktor Albertovich MINKIN, St. Petersburg (RU)

(73) Assignee: OBSHCHESTVO S OGRANICHENNOJ OTVETSTVENNOST`YU "MNOGOPROFILNOE PREDPRIYATIE "ELSYS", St. Petersburg (RU)

(21) Appl. No.: 16/496,887

(22) PCT Filed: Dec. 25, 2017
(86) PCT No.: PCT/RU2017/000964

§ 371 Date: Sep. 23, 2019

(30) Foreign Application Priority Data
Mar. 24, 2017 (RU) 2017109920

(51) Int. Cl. A61B 5/16; A61B 5/11; A61B 5/00

(52) U.S. Cl. CPC A61B 5/165; A61B 5/72; A61B 5/11

(57) ABSTRACT

The invention can be used to obtain information about a psychophysiological state of a person in different fields, including biometrics, psychophysiology, functional diagnostics and psychology. For this purpose, the following primary psychophysiological characteristics are determined: an energy characteristic, as an indicator of the energy given off by a person, and an information characteristic, as an indicator of the efficiency of information exchange. A person's current psychophysiological state is determined in real time, and additionally the pattern of change in the person's current psychophysiological state over a monitoring period is evaluated. The evaluation of a change in the person's psychophysiological state is carried out in a system of coordinates formed by the primary psychophysiological characteristics, where the pattern of change in the person's psychophysiological state over the entire monitoring period is evaluated according to the change in direction of a vector characteristic in the form of the path of a graph consisting of consecutively interconnected directed segments characterizing the change in direction and magnitude of the primary psychophysiological characteristics for each recorded time interval of the monitoring period. The present method provides an increase in the functional possibilities of a method of evaluating a person's psychophysiological state as a result of the additional analysis of psychophysiological characteristics based on information about the pattern of change in the direction of a vector of the person's unconscious response during the monitoring process, and establishment of a correlation between the change in direction of the psychophysiological response vector and a set of primary physiological parameters determining the person's psychophysiological state.

### TECHNICAL FIELD

The present invention relates to the fields of the biometrics, psychophysiology, functional diagnostics, psychology, metrology and can be used to obtain information on psychophysiological, psychosomatic and physiological characteristics of a person, control an emotional state, carry out functional diagnostics of persons and animals, as well as conduct psychological and psychophysiological tests and researches.

### BACKGROUND

Modern methods of obtaining information on a current person's psychophysiological state are mostly based on obtaining the dependence of a particular physiological parameter on time and determining its stability or time variability. Traditional psychophysiological lie detectors [1], as well as most systems of medical and functional diagnostics of a person's psychophysiological state [2, 3], operate based on this principle. This approach allows objective physical methods to measure values of the physiological characteristics of a person and to carry out on their basis a qualitative assessment of certain psychophysiological parameters and a person's state, but does not allow to provide an objective measurement and assessment of the current person's psychophysiological state, because physiological parameters (heart rate (HR), electroencephalogram (EEG), galvanic skin response (GSR)) known and used to be measured are not uniquely linked to the person's psychophysiological or emotional state and are not able to reflect all possible changes in the person's psychophysiological response.

“A method for detecting hidden emotion response on showing an object under investigation” is known in the prior art [4]. This method comprises showing special information (in particular, images), recording a psychophysiological response, analyzing the obtained data, and identifying hidden emotion response by deviation from the norm. Moreover, pulsometry is used to record the psychophysiological responses, and the initial number of pulse beats during the demonstration of images to detect an increase in heart rate is the smallest number of beats detected during the demonstration of images, and not the pulse rate corresponding to the beginning of the study.

According to the invention, the study of recorded psychophysiological responses is carried out by measuring the heart rate, and a conclusion about a person's psychic state is made by the difference between the frequency of the pulse measurements.

However, showing the selected images to a person being tested may cause him psychic responses of a different nature: either responses to the positive emotions of some pleasant events associated with one or more of the images shown; or responses to the negative emotions of unpleasant events associated with another or other images shown. In both cases, there may be a sharp increase in the pulse rate, and the pulse rate with positive emotions may exceed the pulse rate with negative emotions.

The example of this patent illustrates the main problem of the modern detection of the psychophysiological state, in particular, the lack of an unambiguous correlation between the recorded values of physiological parameters and the direction of a psychophysiological response vector of the one being tested. As shown above, it is commonly impossible to determine, based only on changing the physical quantity (value) of the psychophysiological parameter being measured, whether this change is caused by positive or negative emotions. This problem is typical one for all types of modern measurable physiological parameters (EEG, ECG, GSR, etc.). Therefore, it is possible to state that using the proposed known methods of measuring the dependences of the values of individual physiological parameters on time, without taking into account their relationship, it is impossible to objectively assess the current psychophysiological state of the one being tested, that is, it is impossible to unambiguously determine the vector of its emotion response—whether it is positive or negative.

Attempts to quantify when studying a person's psychological type have been made by Hippocrates [5], one of the first researchers of the archaic era, who posed the problem of finding a measure of psychophysiological quantity hidden in the person himself, the characteristics of his psychic and physical organization. The next significant step in assessing the psychological type and psychophysiological state has been made by the founders of analytical psychology by conducting psychological tests or psychometry using thematic questionnaires. The most famous, generally recognized and scientific is a psychometric approach of Hans Eysenck, who proposed a method for calculating personality traits (extraversion and neuroticism) that form the psychometric axes. Eysenck [6] proposed several variants of questionnaires with a quantitative assessment on the scales of “neuroticism” (imbalance in the processes of excitation and inhibition of the nervous system) and “extraversion”-“introversion” (direction of the personality outward or inward), which have found wide practical application. The subjectivity of the questionnaire method forced Eysenck to resort to the introduction of a control group of questions assessing the sincerity of the one being tested, which made it possible to reject data with low reliability.

The claimed method uses two independent parameters to assess a personality state. However, the very principle of obtaining psychophysiological information through a questionnaire is quite subjective and cannot be considered as a physical measurement, since it is not based on a direct measurement of physical quantities in determining the person's psychophysiological response.

In papers of Wilhelm Wundt [6] and James Russell [7], the emotional factor becomes dominant in determining a psychophysiological state. There is a departure from the macro level to the micro level—emotions, a basic affect in which emotion is a measure of a physical quantity. Most models of emotions are two-factor, as well as previously considered models of temperament and personality, the primary parameters of which are the sign of emotion (positive/negative) and the level of activation (high/low). Wundt distinguishes three dimensions of emotions: pleasure-displeasure, calmness-excitement, intenseness-abreaction. Despite attempts at a quantitative approach to assessing emotions and the psychophysiological state, all these researchers relied primarily on psychological rather than physical scales for assessing PPS of a person.

It should also be noted that the number of publications of applications and patents for inventions aimed at developing technical means and methods for analyzing a psychophysiological state and behaviour of a person has recently increased [9, 10, 11]. However, most of the proposed solutions are also based on the measurement of physiological parameters or psychological characteristics of a person, which is insufficient to obtain objective information on the current psychophysiological state and limits the application of the developed methods.

A method for obtaining information on psychophysiological state of living being is known, patent RU 2510238 [12]. This method allows to determine a set of person's psychophysiological characteristics based on the processing of the measured physiological parameters in real time, namely, the parameters of the movements of the head of a living being, obtained by vibraimage technology. This method comprises determining the set of its psychophysiological characteristics, selecting from said set of primary psychophysiological characteristics characterizing a psychophysiological state and determining a current psychophysiological state in real time while mathematical processing measurement data of the primary psychophysiological characteristics. To do this, the processing of the registered signal is performed, which includes the conversion of quantitative parameters of the spatial and temporal distribution of the movement of the head of a living being into information-statistical parameters characterizing the set of the psychophysiological responses of the one being tested.

This method [12] is taken by us as a prototype. The prototype allows to get the set of person's psychophysiological parameters, however, as in the case of the classical polygraph, the prototype records the dependences of the measured psychophysiological parameters on time, which is not informative enough to reveal an unambiguous correlation with the vector of change in the emotional (psychological) state of a person. Such an approach allows to monitor the change in each psychophysiological parameter in time, but does not allow to evaluate the direction of change of the current person's psychophysiological state, the pattern of change in the direction of a vector of his emotion response. For each specific application, it is necessary to establish their local experimentally determined thresholds and relationships between parameters. To diagnose a certain disease or functional state, it is necessary to experimentally establish and confirm formulas and dependencies of the parameters from each other, which significantly limits the breadth of use of the method [10].

### SUMMARY

The objective of the present invention is to develop a universal method of researching and evaluating changes in a current psychophysiological state of a person.

The technical effect of the claimed method of researching and evaluating a person's psychophysiological state (PPS) lies in expanding the functional possibilities of a method of evaluating PPS through an additional analysis of psychophysiological characteristics based on information on the pattern of change in the direction of a vector of the person's psychophysiological (unconscious) response during the monitoring process, as well as improving the accuracy of determination of the psychophysiological state and establishing a correlation between the change in direction of the pcychophysiological response vector characterizing a change of a current psychophysiological state, and a set of primary physiological parameters determining the person's psychophysiological state.

The technical effect is achieved by using a method of evaluating a person's psychophysiological state, the method comprising determining a set of person's psychophysiological characteristics, identifying primary psychophysiological characteristics from the set and determining a current person's psychophysiological state in real time while mathematical processing measurement data of the primary psychophysiological characteristics, that differs from the prototype in that the method further comprises evaluating a pattern of change in the current person's psychophysiological state during the monitoring period, wherein, as the primary psychophysiological characteristics, a characteristic of the physical state is identified, including at least an energy characteristic, as an indicator of the energy given off by a person, and an information characteristic, as an indicator of the efficiency of information exchange, characterizing the change in the level of information exchange within and between person's physiological systems, under the influence of external and internal variable factors, determining measured values of the primary psychophysiological characteristics at each recorded time of the monitoring period, the evaluation of the change in the person's psychophysiological state is carried out in a system of coordinates formed by the primary psychophysiological characteristics, wherein the current person's psychophysiological state at a recorded i-th time is defined as a point located at the intersection of coordinates formed by measured values of the primary psychophysiological characteristics at said recorded time, the change in the current person's psychophysiological state over an i-th recorded time interval of the monitoring period is determined based on direction of a vector of a directed graph segment connecting the intersection points of the coordinates of the primary psychophysiological characteristics at an initial (i−1) and end (i) recorded time, and a pattern of change in the person's psychophysiological state over the entire monitoring period is evaluated according to the change in direction of a vector characteristic in the form of a path of a graph consisting of consecutively interconnected directed segments characterizing the change in direction and magnitude of the primary psychophysiological characteristics over each recorded time interval of the monitoring period.

In a further embodiment of the invention, as one of the primary psychophysiological characteristics determining the energy characteristic of the person being tested, a vibraimage parameter is selected that reflects an average frequency of micro-vibrations of a person's head, and, as another primary psychophysiological characteristic determining the information characteristic of the psychophysiological state of the person being tested, a vibraimage parameter is selected that reflects a scatter (standard deviation) of the frequency of micro-vibrations of the person's head, and carry out their calculation.

In another embodiment of the invention, the change in the current person's psychophysiological state dP is determined according to change in the coordinates of its energy characteristic dE which is determined as dE=Eᵢ₋-Eᵢ, and information characteristic dI which is determined as dI=Iᵢ-Iᵢ₋₁, where:

Eᵢ₋₁—an initial reference coordinate of the energy consumption of the initial person's state at the recorded i-th time interval of the monitoring period;

Eᵢ—an end reference coordinate of the energy consumption of the current person's state at the recorded i-th time interval of the monitoring period;

Iᵢ₋₁—an initial reference coordinate of the information characteristic of the initial person's state at the recorded i-th time interval of the monitoring period;

Iᵢ—an end reference coordinate of the information characteristic of the current person's state at the recorded i-th time interval of the monitoring period.

In a further embodiment of the invention, upon presentation of a stimulus, the change in the current person's psychophysiological state dP may be determined by the formula:

dP=dI+dE,

where:

dE is a change in coordinates of an energy characteristic, determined by the formula:

(dE=Eᵢ₋₁−Eᵢ),

dI is a change in coordinates of an information characteristic, determined by the formula:

(dI=Iᵢ−Iᵢ₋₁),

where:

Eᵢ₋₁—an initial reference coordinate of the energy consumption of the initial person's state at the recorded i-th time interval of the monitoring period;

Eᵢ—an end reference coordinate of the energy consumption of the current person's state at the recorded i-th time interval of the monitoring period;

Iᵢ₋₁—an initial reference coordinate of the information characteristic of the initial person's state at the recorded i-th time interval of the monitoring period;

Iᵢ—an end reference coordinate of the information characteristic of the current person's state at the recorded i-th time interval of the monitoring period.

The present solution is based on the following assumptions.

Since there are psychological, physiological, and behavioral components in each psychophysiological state, then, in the descriptions of the nature of states known from the prior art, one may find the concepts of different sciences (general psychology, physiology, medicine, labor psychology, etc.). Moreover, at present, there is no common point of view on the problem of changes in psychophysiological states, since they are also samples of the personality dynamics, due to relationships, behavioral needs, goals of activity, and adaptability of the personality in the environment and situation. As previously described, modern methods of researching a current person's psychophysiological state are overwhelmingly aimed at evaluating the already existing psychophysiological state without taking into account the pattern, direction, and nature of the change in this state over the monitoring period, which does not allow to reveal the generation of crisis states or the formation of uncontrolled emotions, as well as the creation of prerequisites for their occurrence. The impossibility of evaluating the nature of change in person's psychophysiological state by traditional methods is due to the lack of practice and methods of simultaneous evaluating not only the nature of change in any particular indicator (physiological or psycho-emotional) of a person's state during the researches, but also their relationship, which makes it possible to evaluate only one component of the psychophysiological state, either at the level of psycho-emotional state or emotions, or at the level of physiological (energy) responses. Since these components are inextricably associated with each other, and the change in each of them is associated with a large number of both internal physiological and psycho-emotional changes occurring, as well as external influences on the person: psychological, physical, informational, etc., it is obvious that known methods cannot provide a reliable analysis of current changes in the person's psychophysiological state due to the lack of a complete characteristics of the information impact on a person from different functional systems and changeable external factors, as well as the lack of approaches to objectively measure changes in information exchange between physiological systems of the person.

Meanwhile, the modern scientific approach to a person whose technical analogue (equivalent) is a complex cybernetic or information-measuring system, proposed by the founders of cybernetics [14, 15, 16], offers the use of two basic concepts of “information” (“information exchange”) and “energy” to characterize an object of any complexity. Despite the apparent obviousness of this approach, in terms of characterizing a current person's psychophysiological state, it has not been used to date. Despite the fact that the characteristic of energy consumed by a person (in kcal/min) is quite popular for determining the functional state of a person but in most cases it is used only in dietetics and medicine [17]. The main obstacle to adaptation of the proposed approach to characterizing person's psychophysiological state was the practical non-use in psychology and physiology of the term “information” as such, “information state” (“information exchange”), as well as the term “information efficiency”, as an indicator of information exchange (i.e. an indicator of a change in the level of information exchange within and between physiological systems of a person), or the use of said terminology in a slightly different sense, in relation the founders of cybernetics. For example, the information theory of emotions, developed by academician Simonov [18], suggested an emotional response in response to an external information impact on a person. However, the closest synonym in essence to the term information exchange (or an information exchange indicator), which characterizes the information state of an object (person), is the term psychological state of a person, which determines the state of person's psychological comfort. In terms of sensory physiology [19], any psychic and physiological processes occurring in a person are determined by information interaction through physical and chemical processes in their close relationship [14]. Therefore, the psychic and psychological state of a person may be determined using information characteristics that determine the state of person's psychological comfort, and the physiological state, in turn, may be displayed using energy characteristics. The claimed solution is based on the assumption that the information state of a person and the information exchange characterizing it is determined by information efficiency, i.e. the quality of information exchange (speed, magnitude of signals, losses, signal-to-noise ratio, etc.) of the passage of information signals within and between human physiological systems. This approach is similar to the classical approach of Shannon, Wiener and Bernstein to transmit information for the technical and biological system [14, 15, 16] and is confirmed in the previously mentioned works on the information theory of emotions and sensory physiology.

When considering a person as an abstract physical or cybernetic system, the indicators of person's information state (information exchange) depend on controllability and losses, i.e. on the speed and synchronism of the passage of sensory feedback signals in each of the physiological systems and functionally related physiological systems [11, 13]. Moreover, as is known, according to academician Pavlov, all person's physiological systems are interconnected [20]. In the course of his experimental psychophysiological researches, an author found that in the case of mood improvement and emotional uplift, an increase in the degree of synchronization of the functioning of various physiological systems of a person is observed, and in the case of a deterioration in the person's functional state, said degree of synchronization decreases. For example, the Pearson correlation coefficient determined by processing various physiological signals, for example, heart rate (cardiovascular system), GSR (skin system), vestibulometry (vestibular system), may serve as said information exchange indicator. In this case, the average sum of Pearson's correlation coefficients determined between several different physiological signals reflects the general level of person's controllability or an indicator of person's information state (information exchange). The author experimentally established that another possible information characteristic, which is an indicator of the synchronization of the operation of physiological systems, is a scatter of the frequency of micro-vibrations of the person's head, determined by vibraimage technology.

For greater clarity, an example of the invention is discussed below, in which a person is presented in the form of a conventional cybernetic system (FIG. 1) consisting of a number of physiological systems Ph₁-Phₙ (cardiovascular, nervous, digestion, vestibular, etc.), the operation of each of which has a specific physiological task. Moreover, each of the physiological systems has a certain effect on all other systems (transfers its information and receives corrected information back, in the form of a feedback), the mutual influence of one physiological system on another is characterized by the correlation coefficient Cₖₙ. Input effects (stimuli) in the form of energy carriers Eₑₓ (food, oxygen) and input information Iₑₓ (light, sound, heat, etc.) are constantly fed to a person's “input”, which are converted by the metabolism of internal physiological processes (Iᵢₙ−Eᵢₙ ) into external demonstrations in the form of energy Eₒᵤₜ (heat, movement) given off by the person and information Iₒᵤₜ (words, appearance, changes in physiological parameters of heart rate, GSR, ECG, etc.). Of course, this is a general scheme of person's functioning, however, according to the author's opinion, it is such a scheme that fully covers the physical, chemical and information processes that occur with a person and well explains the invention.

The objective of the invention is to evaluate a person's psychophysiological state which, as follows from the scheme shown in FIG. 1, is inextricably associated with the need to determine changes in internal energy as a result of physicochemical processes Eᵢₙ occurring inside a person and an indicator of changes in person's information state Iᵢₙ characterized by an information exchange. It follows from the scheme that the closest analogues of the internal values of the information and energy state of a person are their external components Iₒᵤₜ and Eₒᵤₜ, which may be physically measured. In this case, the determination of the energy given off or consumed by the person is a well-known physical problem that has many technical solutions. According to the claimed solution of the invention, it is proposed to determine the internal information state as the information efficiency Iᵢₙ=Iᵤ/Iₜ (Iu is useful information, i.e., the amount of information transmitted and received without loss and repetition per unit time; It is the total amount of information transmitted per unit time) or the reduced function of the sum of the correlations of operation of various physiological systems (Iᵢₙ=F(S(Cₖₙ)), and this process may be carried out in various technical ways, for example, using vibraimage technology or by measuring the average correlation of the time dependencies of various physiological signals. A decrease in the useful signal exchange and correlation dependence during the functioning of various person's physiological systems is characterized by a loss of control, chaos, an increase in entropy and, in extreme cases, death of the person.

Obviously, the proposed information-physical approach to the analysis of person's psychophysiological state has a number of undeniable advantages. The parameters traditionally used to characterize the psycho-emotional and psychophysiological state (aggression, stress, pleasure, neuroticism, extraversion) are almost always of subjective nature. Most of existing approaches to the analysis of person's psychophysiological state are based on the well-known statement of the ancient Greek philosopher Protagoras, who claimed that “man is the measure of all things.” However, to obtain objective metrological results when measuring person's psychophysiological state, it is necessary to avoid ambiguous psycho-emotional characteristics, and use only physical quantities and objectively measured technical characteristics.

In the prior art (including in the prototype), as previously noted, technical solutions were distinguished by the fact that, as a rule, one or more time dependent physiological parameters were recorded. At the same time, there was no functional dependence between the parameters being studied and it was impossible to determine a current psychophysiological state on the basis of general mathematical relationships between the parameters. In accordance with the claimed solution, two basic parameters are physically measured that determine the energy and information state of a person, and the psychophysiological state is determined by the ratio of these two parameters, in coordinates: information/energy (I/E). An information indicator of a person's state is understood as a level of controllability (i.e., information efficiency, as a ratio of the received amount of informative signals to the entire amount of transmitted information) or a level of correlation between various physiological systems of a living organism, which, as is known, may be objectively determined, for example, based on correlations between the signals of the electrocardiogram, electroencephalograph, galvanic skin response and vestibulometry, which vary in time and are interconnected. As an energy indicator of a person's state, one should understand an energy physically consumed or given off by a person, which in equilibrium is approximately the same for a long period of time (at least 24 hours), whereas in short periods lasting 5 to 20 seconds, for example, the response period per stimulus, the discharge (release) of person's energy may be several times higher than the energy consumption for the same time, the average energy discharge in a calm state and the average daily energy discharge.

If the value of the parameter reflecting the energy characteristic E of the organism decreases, and the value of the parameter reflecting the information state I of the organism increases, then one may conclude that the person goes into a more silent relaxed state. If, on the contrary, the value of the parameter corresponding to the energy characteristic of the organism increases, and the value of the parameter corresponding to the information state of the organism decreases, then the person is in a state of nervousness, stress. If the values of both parameters E and I increase, then the person goes into an active composed state, ready for action. If the values of both parameters decrease, then the person is in a depressed, heaviness state.

In such a way, two determining parameters of the system appear, by the ratio of which one may unambiguously interpret the change in person's psychophysiological state. We obtain a two-dimensional array of data that respectively determine the information and energy characteristics of the recorded responses (I(t)/E(t)), which array allows to establish a correlation with a change vector of a current psychophysiological state, that is, at least, allows to determine whether this response positive or negative. According to the claimed invention, it is the degree of positivity or negativity of the change in the current PPS under the influence of external and/or internal factors, including when sensing an external stimulus, that are the determining factors for psychophysiology, as 1 and 0 of modern computing technology. Therefore, on the basis of the data obtained, it is possible to draw unambiguous conclusions about the nature of changes in the current person's psychophysiological state in the information and energy scale (%/kcal), as unambiguously and definitely as, for example, a current-voltage characteristic of a transistor is measured.

One may say that this approach is somewhat close to Eysenck's assessment of personality on the scales of “extraversion”-“neuroticism”, since it is possible to draw a parallel between the energy characteristic of a person's state and the degree of extraversion of his behavior, as well as the level of psychological comfort and the degree of his poise (neuroticism). However, as noted above, the Eysenck test is based only on questionnaires, whereas, in accordance with the claimed solution, the assessment is based on actually measured physical data.

One of the technologies suitable for implementing the claimed method is vibraimage technology which allows to measure both person's energy consumption and indicators of information state. The author has experimentally established that a scatter of a vibraimage frequency characterizes an information indicator of the person's psychophysiological state, and an average frequency of vibraimage characterizes its energy indicator.

Obviously, for the compatibility of processes of scaling estimates of information and energy characteristics of a person, they may be expressed in physical units (information efficiency in percent, energy in joules or calories, and for a recorded period of time—power in J/min or kcal/min) or both characteristics may have relative indicators (%), in this case, the energy characteristic should be reduced to person's ultimate capabilities.

Then, on the basis of the foregoing, as an indicator of an information characteristic (information efficiency) of a person, the relation of information exchange between and within physiological systems of a person to the total amount of signal exchange between and within physiological systems may be taken, as follows:

Iin=Ir/(Ir+S); (1)

where

Iin—a characteristic of the person's information state;

Ir—an amount of useful information accepted by person's physiological systems per unit of time;

Ir+S—a total amount of information sent by person's physiological systems per unit of time (including errors and lost information or entropy S)

Moreover, a reduced indicator of a person's energy state may be expressed by the following proportion:

Ein=Ecur/Emax (2)

where

Ein—a reduced indicator of a person's energy state;

Ecur—an amount of energy consumed by a person per unit of current time;

Emax—a physiological limit of the maximum amount of energy consumed by a person per unit of time.

The above formulas for calculating the reduced indicators of the information and energy characteristics explain the general meaning of these indicators. Obviously, it is impossible to accurately measure the total amount of information transmitted by a person per unit time, because one person's brain containing approximately 50×10¹⁰ neurons [21], transmits more information than all computers in the world, however, the author experimentally established the possibility of evaluating, using vibraimage technology, the ratio of the useful amount of information transmitted by physiological systems to the total amount of information transmitted, as well as a current energy consumed by physiological systems to the limit energy, thanks to the vestibular-emotional reflex.

However, using a similar approach, the initial characteristics of the psycho-emotional state for the claimed method may be obtained using other technologies, such as ECG, GSR, EEG, etc. To do this, it is necessary to measure an energy gven off by a person, for example, using a thermal imager, and evaluate a total synchronism (Pearson correlation coefficient) of the received physiological signals of the ECG, GSR, EEG. etc.

In connection with the foregoing, the technical solution is new, not obvious from the prior art for an average person skilled in the art, and also industrially applicable, therefore, the invention meets the patentability criteria established for inventions.

### BRIEF DESCRIPTION OF DRAWINGS

FIG. 1 shows a general scheme of person's functioning in the form of a conditional equivalent cybernetic system

FIG. 2 shows a scheme of an information and energy diagram of changes in the psychophysiological state.

FIG. 3 shows an example of a real information and energy diagram of changes in the psychophysiological state during the survey.

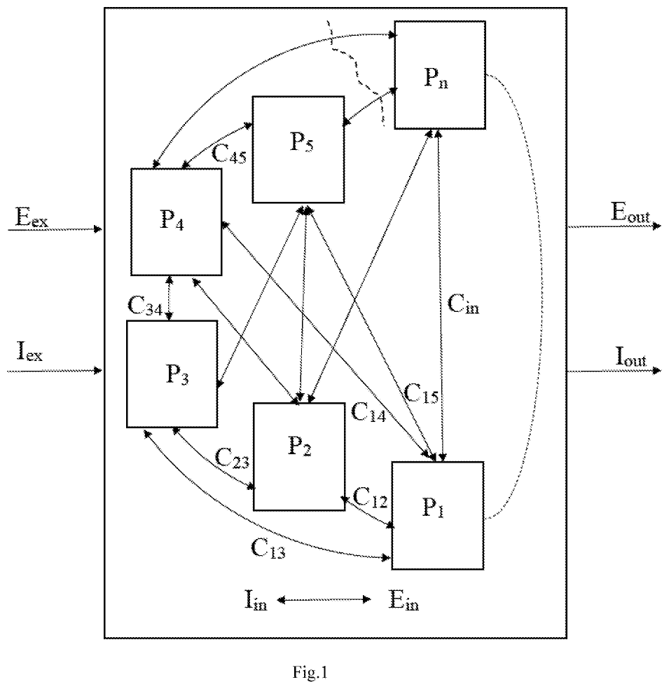

### DETAILED DESCRIPTION OF EXAMPLE EMBODIMENTS

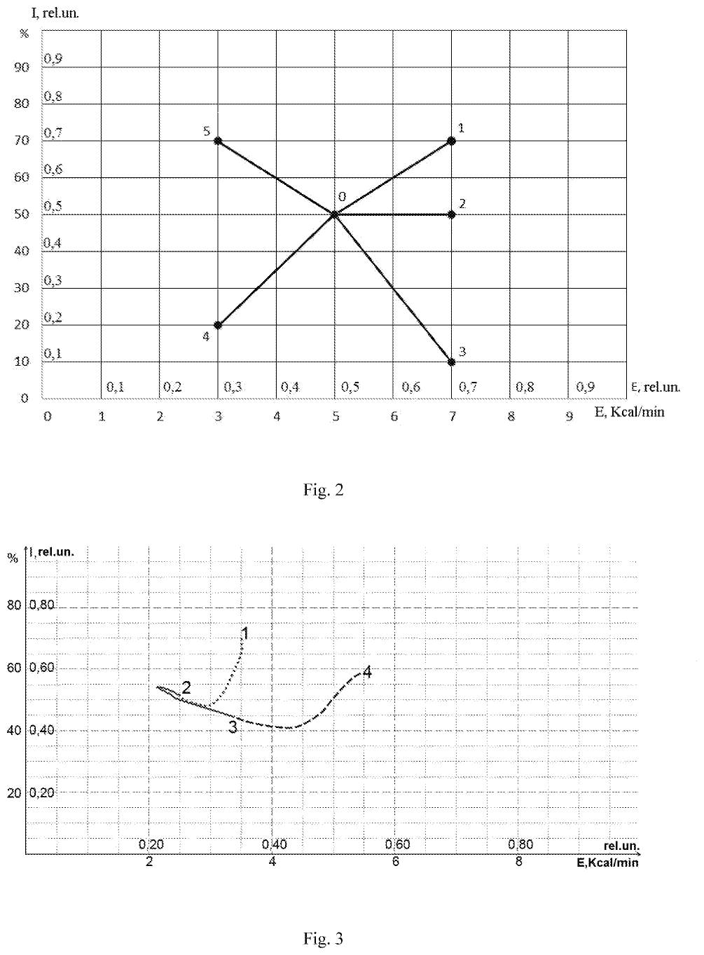

An example of the invention is considered below, in which a change in a person's energy state (an amount of energy consumed (in physics, power) in kcal/min) occurs by a fixed amount (2 kcal/min), and an information parameter characterizing a level of psychological comfort changes in different directions. A current psychophysiological state is displayed in the form of a point lying at the intersection of coordinates formed by measured values of the primary psychophysiological characteristics, and the change in the person's psychophysiological state over time is displayed as a directed segment of a straight line or a curve between two points fixing a person's psychophysiological state at the beginning and end of a studied time interval. This example of a change in the psychophysiological state from state 0 to various states 1, 2, 3, 4, 5 is shown in FIG. 2. In the information and energy diagram shown in FIG. 2, the transition from an initial psychophysiological state (point 0) to other states is characterized by the same change in energy consumption, but different directions of change in the information parameter and psychological comfort. The cause-effect relationships of changes in the psychophysiological state shown in FIG. 2 are discussed below.

Transition 0-1 is characterized by an increase in the energy consumption and an increase in the level of an indicator of an information state (efficiency) and psychological comfort. The reason for this change in the psychophysiological state may be a good or pleasant news (stimulus), which leads to more intense metabolic processes (an increase in energy being consumed), while the psychological state and mood improved significantly (an entropy of metabolic processes decreased, and an information content of the exchange increased).

Transition 0-2 is characterized by an increase in the energy consumption and a constant level of the information state indicator, its efficiency, and psychological comfort. The reason may be, for example, an increase in mental or physical activity, which leads to more intense metabolic processes (an increase in energy being consumed), while the psychological state and mood remain unchanged, since the effect performed did not cause emotional changes.

Transition 0-3 is characterized by an increase in the energy consumption and a decrease in the level of the information state indicator and psychological comfort. The reason may be unpleasant information which leads to more intense metabolic processes (an increase in energy being consumed), while the psychological state and mood noticeably deteriorated (the entropy of metabolic processes deteriorated, and the information content of the exchange decreased, and therefore the efficiency of the information exchange decreased).

Transition 0-4 is characterized by a decrease in the energy consumption and a decrease in the level of the information state indicator and psychological comfort. The reason may be an unpleasant news which leads to a slowdown in metabolic processes (a decrease in energy being consumed), while the psychological state and mood noticeably deteriorated (the entropy of metabolic processes deteriorated, and the information content of the exchange decreased, i.e., the efficiency of the information exchange also decreased).

Transition 0-5 is characterized by a decrease in the energy consumption and an increase in the level of the indicator of information state (efficiency) and psychological comfort. The reason may be a good news which calms and leads to a slowdown in metabolic processes (a decrease in energy being consumed), while the psychological state and mood improved significantly (the entropy of metabolic processes decreased, and the information content of the metabolism increased).

In all the examples provided the change in energy being consumed by a person from state 0 to another psychophysiological state is 2 kcal/min, with each transition having its own emotional and psychophysiological meaning, however, it is physically impossible to determine this meaning by the time dependence of the physiological parameter (GSR, heart rate, EEG, vibraimage and etc.). Therefore, the lack of information about the vector of changes in the person's psychophysiological state, including his emotional state, makes the use of the classic polygraph and similar technologies an art that depends on an operator conducting the test, and does not give reproducible objective results, while the present invention allows to measure and calculate a current psychophysiological state by determining the direction of change in the psychophysiological state.

In the example of the present invention, implemented in practice, the measurement of the current psychophysiological state is carried out by a vibraimage system (Vibraimage PRO) [21] manufactured by Elsys, St. Petersburg, Russia. The vibraimage system measures the current person's psychophysiological state in synchronization with the presented stimuli (visual, textual, graphic and audio information) and, in accordance with the test methodology of Baxter's comparison zones [18], analyzes the sincerity of the one being tested when answering the questions presented. An example of a real information and energy diagram of changes in the psychophysiological state during the survey is shown in FIG. 3. It shows the dependence of the current person's psychophysiological state when answering questions asked. Section 1-2 shows a response of the one being tested when presenting a neutral question, section 2-3 shows the response of the one being tested when presenting a control question, and section 3-4 shows the response of the one being tested when presenting a relevant question. It is interesting to note that in this example, the psychophysiological response of the one being tested was different in direction when answering various categories of questions. In addition, it follows from the above dependence that the psychophysiological response has a certain inertia, since the direction of change in the psychophysiological state does not occur immediately after the presentation of the question (stimulus).

Conducted comparative tests of the system based on the present invention, in which the level of the psychophysiological response of the one being tested to the presented stimulus was determined by the formula (3):

dP=dI+dE (3)

where: dE is a change in coordinates of an energy characteristic, determined by the formula: (dE=Eᵢ₋₁−Eᵢ); dI is a change in coordinates of an information characteristic, determined by the formula: (dI=Iᵢ−Iᵢ₋₁), where Eᵢ₋₁—an initial reference coordinate of the energy consumption of the initial person's state at the recorded i-th time interval of the monitoring period; Eᵢ—an end reference coordinate of the energy consumption of the current person's state at the recorded i-th time interval of the monitoring period; Iᵢ₋₁—an initial reference coordinate of the information characteristic of the initial person's state at the recorded i-th time interval of the monitoring period; Iᵢ—an end reference coordinate of the information characteristic of the current person's state at the recorded i-th time interval of the monitoring period;

showed a 30% reduction in the probability of errors in determining lies in answers to questions regarding a similar analysis of answers to identical questions, taking into account only the energy component of the response of the one being tested, determined in a standard way based on time dependences of the vibraimage parameters.

The provided example clearly demonstrates an increase in accuracy of determining the current person's psychophysiological state in comparison with the registration of one-dimensional changes in the time dependences of person's physiological parameters, wherein, in the provided example, the value of the change in the PPS has a magnitude and a sign proportional to the level of positivity (+) or negativity (−) in response to external and internal influences, to the stimulus. The said example of a specific embodiment shows the practicability of the invention, but the implementation of the method is not limited to the examples provided. The claimed method may be used not only for the psychophysiological detection of lies, but also for many other applications in which it is necessary to measure a current person's psychophysiological state, for example, conducting interviews, surveys, studying a response of the one being tested to various factors, including analysis of the effect of advertising on a person, compatibility checks, pre-shift psychophysiological control, psychological studies of groups of people, etc. The claimed method may be implemented not only on the basis of vibraimage technology, but also with various technologies for measuring physiological parameters, if these parameters allow the psychological (information) and physiological (energy) components to be distinguished from the psychophysiological response. The vibraimage technology also provides the possibility of forming more than two psychophysiological coordinate axes. However, practice shows that in many cases simpler models for determining the psychophysiological state turn out to be more efficient. It should not be understood that the claimed method for determining a current person's psychophysiological state denies the possibility of determining individual psycho-emotional characteristics, such as aggression, stress, anxiety, extroversion, etc. On the contrary, a real measurement of these emotions and psychophysiological characteristics becomes possible only with the use of this method, taking into account the position and the vector of changes in the information and energy characteristics of a person.

### REFERENCES

1. Alekseev L. G. Psychophysiology of lie detection. Methodology. M, 2011, 108 pages.

2. RU 2214166, Device for determining human psychophysiological condition, published on 20 Oct. 2003, Berezhnoj V. N., Bryksin V. N., Talalaev A. A.

3. RU 2246251, Method for evaluating psychophysiological state according to human cardiac rhythm, published on 20 Feb. 2005, Godunov V. A. et al.

4. RU 2036608, Method for detecting hidden emotion response on showing an object under investigation, Viljumanis J. N.

5. Hippocrates. Selected books. Translation from Greek Rudnev V. I. (Moscow-Leningrad: Biomedgiz, 1936.—Series “Classics of Biology and Medicine”).

6. Eysenck H., Wilson G. Know your own personality. Temple Smith, 19757. Wundt W. Introduction to Psychology, George Allen And Unwin, Limited, 1924

8. Russell J. A. Circumplex model of affect, Journal of Personality and Social Psychology. 1980. Vol. 39, No 6, pages 1161-1178.

9. U.S. Pat. No. 9,380,976, Optical neuroinformatics, published on 5 Jul. 2016, Matthew E. Stack.

10. U.S. Pat. No. 8,622,901, Continuous monitoring of stress using accelerometer data, published on 7 Jan. 2014, Jawahar Jain et al.

11. EP 1871219 Methods and systems for physiological and psycho-physiological monitoring and uses thereof, published on 2 Jan. 2008, Tuvi Orbach.

12. Prototype, RU 2510238, Method for obtaining information on psychophysiological state of living being, published on 27 Mar. 2014, Minkin V. A.

13. RU 2515149, Screening diagnostic technique for prostate cancer, published on 10 May 2014, Blank M. A. et al.

14. Weiner N. Cybernetics or Control and communication in the animal and machine. Paris, (Hermann & Cie) & Camb. Mass. (MIT Press), 1948

15.Shannon, C., A Mathematical Theory of Communication”. Bell System Technical Journal, 1948

16. Bernstein, N., The Coordination and Regulation of Movements. Pergamon Press, Oxford, 1967

17. A guide to assessing physical activity using accelerometry in cancer patients J. M. Broderick & J. Ryan & D. M. O'Donnell & J. Hussey. Springer-Verlag Berlin Heidelberg 2014.

18. Simonov P. V., Selected Works in 2 vols., M., Science, 2004.

19. Tamar H. (1972). Principles of sensory physiology. Published by Thomas Springfield, Ill.

20. 82. Pavlov I. P. (1927). Conditioned Reflexes: An Investigation of the Physiological Activity of the Cerebral Cortex. Translated and Edited by G. V. Anrep. London: Oxford University Press.

21. Suzana Herculano-Houzel, The human brain in numbers: a linearly scaled-up primate brain, Front. Hum. Neurosci., 9 Nov. 2009, Instituto de Ciências Biomédicas, Universidade Federal do Rio de Janeiro, Rio de Janeiro, Brasil

22. Manual of the human psychophysiological state monitoring system, Vibralmage8PRO, publication ELSYS. August 2016, published on August 2016. http://www.psymaker.com/downloads/V18_ManualRus.pdf

23. Backster, C. (1963). Polygraph professionalization through technique standardization. Law and Order, 11, pages 63-64.

1. A method of evaluating a psychophysiological state of a person, comprising determining a set of person's psychophysiological characteristics, identifying primary psychophysiological characteristics from the set and determining a current person's psychophysiological state in real time while mathematical processing measurement data of the primary psychophysiological characteristics, characterized in that the method further comprises evaluating a pattern of change in the current person's psychophysiological state during the monitoring period, wherein, as the primary psychophysiological characteristics, a characteristic of the physical state is identified, including at least an energy characteristic, as an indicator of the energy given off by a person, and an information characteristic, as an indicator of the efficiency of information exchange, characterizing the change in the level of information exchange within and between person's physiological systems, under the influence of external and internal variable factors, determining measured values of the primary psychophysiological characteristics at each recorded time of the monitoring period, the evaluation of the change in the person's psychophysiological state is carried out in a system of coordinates formed by the primary psychophysiological characteristics, wherein the current person's psychophysiological state at a recorded i-th time is defined as a point located at the intersection of coordinates formed by measured values of the primary psychophysiological characteristics at said recorded time, the change in the current person's psychophysiological state over an i-th recorded time interval of the monitoring period is determined based on direction of a vector of a directed graph segment connecting the intersection points of the coordinates of the primary psychophysiological characteristics at an initial (i−1) and end (i) recorded time, and a pattern of change in the person's psychophysiological state over the entire monitoring period is evaluated according to the change in direction of a vector characteristic in the form of a path of a graph consisting of consecutively interconnected directed segments characterizing the change in direction and magnitude of the primary psychophysiological characteristics over each recorded time interval of the monitoring period.

2. The method of claim 1, characterized in that a vibraimage parameter reflecting an average frequency of micro-vibrations of a person head is selected as one of the primary psychophysiological characteristics determining the energy characteristic of the person being tested, and a vibraimage parameter reflecting scatter of the frequency of micro-vibrations of the person head is selected as another primary psychophysiological characteristic determining the information characteristic of the psychophysiological state of the person being tested, and carry out their calculation

3. The method of claim 1, characterized in that the change dP in the current person's psychophysiological state is determined according to change in the coordinates of its energy characteristic dE which is determined as dE=Eᵢ₋₁-Eᵢ, and information characteristic dI which is determined as dI=I₁−Iᵢ₋₁ where:
Eᵢ₋₁—an initial reference coordinate of the energy consumption of the initial person's state at the recorded i-th time interval of the monitoring period;
E₁—an end reference coordinate of the energy consumption of the current person's state at the recorded i-th time interval of the monitoring period;
Iᵢ₋₁—an initial reference coordinate of the information characteristic of the initial person's state at the recorded i-th time interval of the monitoring period;
Iᵢ—an end reference coordinate of the information characteristic of the current person's state at the recorded i-th time interval of the monitoring period

4. The method of claim 3, characterized in that, upon presentation of a stimulus, the change dP in the current person's psychophysiological state is determined by the formula dP=dI+dE , where dE is the change in the coordinates of the energy characteristic, and dI is the change in the coordinates of the information characteristic.
